(19) United States
(12) Patent Application Publication
(10) Pub. No.: US 2026/0150849 A1
(43) Pub. Date: Jun. 4, 2026

(54) LAGENIDIUM JURACYAE WITH BURSAPHELENCHUS XYLOPHILUS (B. XYLOPHILUS)-KILLING ACTIVITY AND USE THEREOF IN CONTROL OF B. XYLOPHILUS

(71) Applicant: Zhejiang A&F University, Hangzhou City (CN)

(72) Inventors: Kai GUO, Hangzhou City (CN); Huoyuan XIANG, Hangzhou City (CN); Wei ZHENG, Hangzhou City (CN); Jianfeng GU, Hangzhou City (CN); Huipeng JIANG, Hangzhou City (CN); Siqi TANG, Hangzhou City (CN)

(21) Appl. No.: 18/968,265

(22) Filed: Dec. 4, 2024

(51) Int. Cl. A01N 63/30; A01N 25/04; A01P 5/00; C12N 1/14

(52) U.S. Cl. CPC A01N 63/30; A01N 25/04; A01P 5/00; C12N 1/14

(57) ABSTRACT

Provided are Lagenidium juracyae with a Bursaphelenchus xylophilus (B. xylophilus)-killing activity and a use of the Lagenidium juracyae in control of B. xylophilus. The Lagenidium juracyae has a strain number of NL03 and an accession number of CGMCC NO. 40587. In the present application, an oomycete Lagenidium sp. NL03 with a B. xylophilus-killing ability is isolated from dead soil nematodes. The biological and molecular identification and the biocontrol potential investigation are conducted for Lagenidium sp. NL03 to provide a reference for the expansion of a biological control resource pool for B. xylophilus and the eco-friendly control of pine wilt disease (PWD).

### REFERENCE TO SEQUENCE LISTING

A computer readable XML file entitled “GWP20240906403_sequence listing”, which was created on Nov. 21, 2024, with a file size of about 18,750 bytes, contains the sequence listing for this application, has been filed with this application, and is hereby incorporated by reference in its entirety.

### TECHNICAL FIELD

The present application relates to the field of biocontrol agents, and specifically to Lagenidium juracyae with a Bursaphelenchus xylophilus (B. xylophilus)-killing activity and a use thereof in control of B. xylophilus.

### BACKGROUND

Pine wilt disease (PWD), also known as pine wood nematode disease, is a disease occurring when B. xylophilus parasitizes in a Pinus plant to cause disorder of physiological activities in the Pinus plant and make the Pinus plant wilt and die. Since PWD came into China in 1982, PWD has caused a severe impact on the ecological environment in China due to its characteristics such as rapid spread and difficult control. A large number of studies have been conducted for the control of PWD inside and outside China. For example, longhorn beetles are directly killed with chemical pesticides or trees dying of PWD are cut down and burned to curb the spread of longhorn beetles, which greatly causes a tremendous resource consumption and is easy to cause the emergence of drug-resistant pathogens. Therefore, the biological control has received more and more attention.

The biocontrol agents currently investigated for PWD mainly include biocontrol bacteria such as Pasteuria penetrans and Bacillus cereus and biocontrol fungi such as Esteya vermicola and Myrothecium verrucaria. The biocontrol agents reduce the morbidity of plants mainly by preventing the occurrence of a disease, and are conducive to reducing the chemical pollution and maintaining the ecological balance. There have been very mature commodities as nematocidal biocontrol agents, with a long history of use. These nematocidal biocontrol agents control the number and population reproduction of nematodes mainly by inhibiting plant parasitic nematodes. The biological pesticide Econem is a biocontrol agent developed with Bacillus penetrans as a main raw material, which can have a control effect on a variety of nematodes. The biological pesticide Econem has been used in many countries. The Bacillus preparation BioNem is provided to control root-knot nematodes and other nematodes. The biological pesticides for controlling PWD mainly include living microorganism pesticides and microbial metabolite pesticides. The microbial agent developed from lethal toxin-producing Bacillus thuringiensis JCK-1233 has been used to control PWD through trunk injection and foliar spray. The two nematocidal compounds of 2-coumaranone and cyclo-(Phe-Pro) isolated from the rhizosphere bacterium Lysinimonas M4 of jack pine exhibit a significant nematocidal activity against pine wood nematode (PWN). In addition, the two compounds have a significant inhibitory effect on breeding egg hatching, feeding, and reproduction.

### SUMMARY

The current studies on oomycetes are mostly focused on the control of pathogenic bacteria, and the studies on a nematocidal potential of oomycetes are far from enough. In the present application, an oomycete Lagenidium sp. NL03 with a B. xylophilus-killing ability is isolated from dead soil nematodes. The biological and molecular identification and the biocontrol potential investigation are conducted for Lagenidium sp. NL03 to provide a reference for the expansion of a biological control resource pool for B. xylophilus and the eco-friendly control of PWD.

The present application provides Lagenidium juracyae with a B. xylophilus-killing activity, where the Lagenidium juracyae has a strain number of NL03 and an accession number of CGMCC NO. 40587.

Morphological Characteristics of this Strain are as Follows:

On 2% Sabouraud dextrose agar (SDA), Lagenidium sp. NL03 grows into colorless or pale-yellow colonies, there are basically no aerial hyphae, and these colonies are irregularly radial and have undulating wrinkles. Hyphae are septate or non-septate hyphae, and are specialized to produce spherical chain segments. Spores in broken spherical hyphae can continue to germinate to produce new hyphae. The production of sporangia can be induced with a spore induction solution, and after the induction, sporangia with a diameter of 35 μm to 50 μm are produced from the cytoplasm and nucleus through a transparent outlet tube. Each sporangium has 30 to 40 kidney-shaped amphitrichous zoospores of 10 μm, and active zoospores are encapsulated by a transparent sporangium wall. After zoospores are mature, the zoospores break through the sporangium wall, are released, and then further swim for about 30 min, and are finally encapsulated to produce spherical spores. Germ tubes are gradually germinated out of the spores to produce slender hyphae.

Cultivation characteristics of this strain: The strain grows the fastest at 30° C., and does not grow at 37° C., indicating that the strain cannot grow in homeothermic mammals.

Molecular biology characteristics of this strain: Nucleotide sequences of internal transcribed spacer (ITS) and three exons (cytochrome oxidase II (COXII), heat shock protein 90 (HSP90), and β-tubulin (β-TUBB)) are shown in SEQ ID NO: 9, SEQ ID NO: 10, SEQ ID NO: 11, and SEQ ID NO: 12, respectively.

The strain isolated and identified in the present application is Lagenidium juracyae with a strain number of NL03, which was deposited in the China General Microbiological Culture Collection Center (CGMCC) at NO. 1 West Beichen Road, Chaoyang District, Beijing, China on May 8, 2023, with an accession number CGMCC NO. 40587.

The present application also provides a use of the Lagenidium juracyae in preparation of a B. xylophilus-killing pesticide.

The present application also provides a biocontrol agent for killing B. xylophilus, including a spore suspension of the Lagenidium juracyae as an active ingredient.

The present application also provides a use of the Lagenidium juracyae or the biocontrol agent in control of PWD in a forest tree.

Optionally, the forest tree is a pine tree.

The present application also provides a preparation method of the biocontrol agent, including:

activating the Lagenidium sp. NL03 to obtain activated Lagenidium sp. NL03, transferring the activated Lagenidium sp. NL03 to an egg yolk+milk agar medium, and cultivating to produce mycelia; transferring a part of the mycelia to a Brey liquid medium for inducing oogonia, and further cultivating for 2.5 d to 3.5 d (preferably 3 d) to obtain a hypha solution; and mixing the hypha solution with a spore induction solution, and cultivating at 28° C. to 30° C. (preferably 30° C.) for 6 h to 10 h.

Optionally, a preparation process of the Brey liquid medium includes: mixing 2.8 g/L of a yeast extract, 2.4 g/L of glucose, 3.2 g/L of a wheat germ powder, and 0.1 mL/L of cottonseed oil to obtain a first composition; mixing 5.0 g of a cottonseed meal with 100 mL of distilled water for 1 h at room temperature (25° C.), and filtering through a cheesecloth to obtain a second composition; and adding 100 mL of the second composition to 900 mL of the first composition.

Optionally, a preparation process of the spore induction solution is as follows: adding 66.04 g of (NH₄)₂HPO₄, 68.05 g of KH₂PO₄, and 87.09 g of K₂HPO₄ to 500 ml of water to obtain a first solution; adding 18.38 g of CaCl₂·2H₂O and 25.42 g of MgCl₂ 6H₂O to 250 mL of water to obtain a second solution; autoclaving the first solution and the second solution separately to obtain a sterilized first solution and a sterilized second solution, and storing the sterilized first solution and the sterilized second solution at room temperature until use; and mixing 0.5 mL of the sterilized first solution, 0.1 mL of the sterilized second solution, and 1.0 L of sterile distilled water to obtain the spore induction solution.

The present application also provides a control method for B. xylophilus, including: impregnating the B. xylophilus with a spore suspension of the Lagenidium juracyae or applying the spore suspension of the Lagenidium juracyae to a forest tree.

Optionally, the spore suspension is a spore suspension when zoospores are released and swim.

Optionally, the B. xylophilus is a 3rd to 4th instar larva.

Optionally, the B. xylophilus is a 4th instar larva.

Optionally, a concentration of the spore suspension is 1×10⁴/mL to 10×10⁴/mL; and the impregnating is conducted for 24 h to 120 h.

Optionally, the forest tree is a pine tree. Further optionally, the forest tree is a pine tree infected with PWD or a pine tree not infected with PWD.

The Lagenidium juracyae or the biocontrol agent prepared based on this strain in the present application can well control PWD when applied to a pine tree infected with PWD, and can well prevent PWD when applied to a pine tree not infected with PWD.

Optionally, the Lagenidium juracyae or the biocontrol agent prepared based on this strain may be applied in a variety of ways, such as common spraying, trunk injection, or irrigation.

Optionally, for the application directly to B. xylophilus, the biocontrol agent prepared from the Lagenidium juracyae is preferred.

Compared with the prior art, the present application at least has the following beneficial effects:

(1) In the present application, an oomycete Lagenidium sp. NL03 is isolated from a soil nematode carcass. It has been discovered through an experiment that the oomycete Lagenidium sp. NL03 has an obvious B. xylophilus-killing ability.

(2) The Lagenidium sp. NL03 is subjected to sequence alignment and phylogenetic analysis, and corresponding results show that the Lagenidium sp. NL03 has the most close genetic relationship to Lagenidium juracyae in terms of ITS and COXII sequences. Multi-gene coalescent tree inference is conducted for the Lagenidium sp. NL03, and corresponding analysis results show that the Lagenidium sp. NL03 is a separate branch on the phylogenetic tree and has a close relationship to Lagenidium humanum evolutionarily.

(3) In the study on infectivity of the Lagenidium sp. NL03, it has been discovered that the Lagenidium sp. NL03 has a strong nematocidal ability and a cytoplasmic extract and a spore suspension of the Lagenidium sp. NL03 can significantly inhibit the hatching of eggs and affect the activity of nematodes. It can be reasonably speculated that the Lagenidium sp. NL03 can produce a specified substance to paralyze the nerves of nematodes and hinder the movement of nematodes, thereby facilitating the infection or providing conditions conducive to the survival of the strain itself. It can be known through an attraction test that nematodes exhibit different tropisms to the Lagenidium sp. NL03 and Botrytis cinerea (B. cinerea).

(4) Lagenidium giganteum is the only Lagenidium isolated from infected mosquito larvae, but two isolates are isolated from Lagenidium giganteum. Among the two isolates, one is a heat-resistant isolate that infects mammals, and the other one is a heat-sensitive isolate that infects mosquito larvae in the nature. The only phenotypic difference between the two isolates is that the two isolates exhibit different tolerances under growth at different temperatures. Over the past two decades, possible infections caused by Lagenidium in mammals have been reported more and more frequently, including infections in cats, dogs, and humans. Therefore, in the present application, the Lagenidium sp. NL03 is cultivated at 25° C., 30° C., and 37° C., and results can preliminarily demonstrate that the Lagenidium sp. NL03 cannot grow in homeothermic mammals. Because Lagenidium juracyae with the most close genetic relationship to the Lagenidium sp. NL03 is isolated from Lagenidium giganteum, for the sake of the safety of aquatic organisms, the present disclosure also attempts to conduct an experiment of a spore suspension of the Lagenidium sp. NL03 on larvae and embryos of Danio rerio. Results of the experiment show that the Lagenidium sp. NL03 basically cannot infect the larvae and embryos of Danio rerio and does not affect the hatching of embryos.

(5) The critical timing of the Lagenidium sp. NL03 to infect B. xylophilus is when zoospores are released and swim. The zoospores adhere to nematodes through indeterminate swimming and penetrate through body walls of the nematodes, so as to allow the purpose of infecting the nematodes. Experiments have shown that the Lagenidium sp. NL03 exhibits selectivity for B. xylophilus during infection, indicating that there are some genes in this strain to regulate this process. Subsequently, related genes are determined by analyzing transcriptome data of an interaction between the Lagenidium sp. NL03 and B. xylophilus. From the perspective of host sources, the Lagenidium sp. NL03 can infect B. xylophilus and has the potential to become a biocontrol agent for controlling PWD.

### BRIEF DESCRIPTION OF THE DRAWINGS

FIG. 1 shows growth rates of Lagenidium sp. NL03 at three temperatures;

FIGS. 2A-21 show morphologies of hyphae of the Lagenidium sp. NL03;

FIGS. 3A-3B show phylogenetic trees constructed according to ITS and COXII of the strain;

FIG. 4 shows a result of multi-gene coalescent tree inference for ITS, COXII, HSP90, and β-TUBB sequences of the strain;

FIGS. 5A-5B show antagonism results between the Lagenidium sp. NL03 and B. cinerea;

FIG. 6 shows infection results of spore suspensions of the Lagenidium sp. NL03 at different concentrations for B. xylophilus;

FIGS. 7A-7B show images illustrating the infection of the Lagenidium sp. NL03 into B. xylophilus;

FIG. 8 shows infection results of the Lagenidium sp. NL03 into other nematodes;

FIG. 9 shows impacts of a spore suspension and a cytoplasmic extract on the hatching of B. xylophilus eggs;

FIG. 10 shows infection results of a spore suspension for B. xylophilus at different instars; and

FIG. 11 shows impacts of a spore suspension on the motility of B. xylophilus at different instars.

### DETAILED DESCRIPTION OF THE EMBODIMENTS

The technical solutions of the present application are clearly and completely described below with reference to embodiments. Apparently, the described embodiments are merely some rather than all of the embodiments of the present application. All other embodiments obtained by a person of ordinary skill in the art based on the embodiments of the present application without creative efforts shall fall within the protection scope of the present application.

Unless otherwise defined, all technical and scientific terms used herein have the same meaning as commonly understood by those skilled in the technical field of the present disclosure. The terms used in the specification of the present application are merely for the purpose of describing specific embodiments, rather than limiting the present application.

Preparation of 2% SDA: 8 g of glucose, 4 g of peptone, 4 g of a yeast extract, and 6 g of an agar are mixed, diluted to 400 mL, and autoclaved at 115° C. for 15 min.

### Example 1 Isolation of a Strain

Hyphae or monospores were picked with sterilized tweezers or a sterilized inoculation needle directly from a body surface of a nematode that had been infected with and killed by an oomycete, transferred to a suitable fungous medium, and cultivated. According to comparison and screening, the most suitable medium was 2% SDA, which was prepared as follows: 8 g of glucose, 4 g of peptone, 4 g of a yeast extract, and 6 g of an agar were mixed, diluted to 400 mL, and autoclaved at 115° C. for 15 min. Oomycetes on the body surface of and inside the nematode were isolated. Whether the oomycetes inside and outside the nematode were of a same species was determined. The Oomycetes were cultivated at about 25° C. Since oomycetes generally grew slower than bacteria, 100 μg/mL of ampicillin could be added to a medium to control bacterial contamination. Strains isolated were purified several times, and screened to finally obtain a strain that could grow on 2% SDA for a long time without contamination by miscellaneous strains as an isolated strain, and the isolate was further identified.

### Example 2 Identification of the Strain

### 1. Isolation and Cultivation of Oomycetes

### Normal Cultivation:

The isolated strain (from Example 1) was cultivated on 2% SDA at 25° C., 30° C., and 37° C. for 7 d, and a colony diameter was continuously measured to determine the optimal growth temperature.

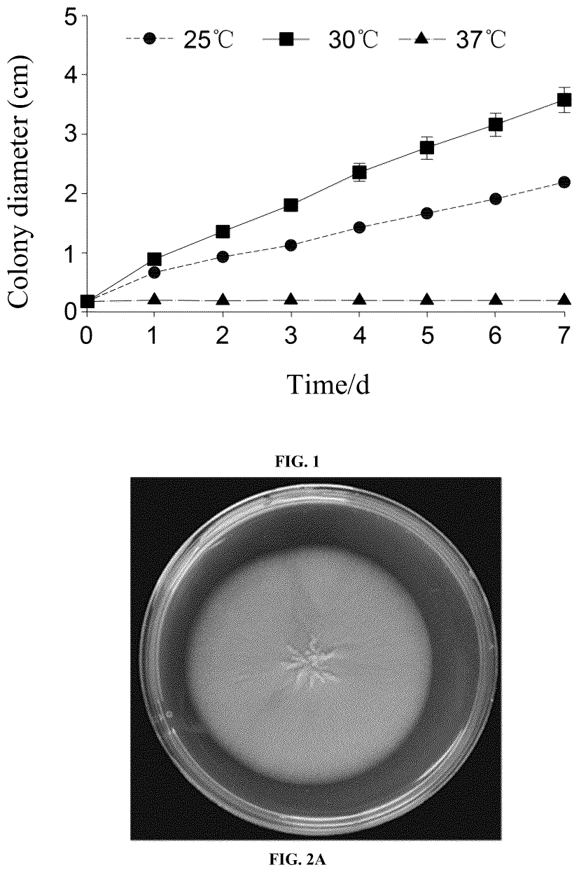

Growth rates of the isolated strain Lagenidium sp. NL03 at the three temperatures were shown in FIG. 1. According to analysis results of colony diameters of the isolated strain Lagenidium sp. NL03 cultivated at 25° C., 30° C., and 37° C. for 7 d, the isolated strain grew the fastest at 30° C., and did not grow at 37° C., indicating that the isolated strain could not grow in homeothermic mammals.

### Induced Cultivation of Oogonia and Zoospores:

(1) A fungal mass sub-cultivated on a 2% SDA plate at 30° C. was picked, transferred to an egg yolk+milk agar medium (the egg yolk+milk agar medium was prepared as follows: one fresh egg yolk per liter, 20 mL/L of skimmed milk, 20.0 g/L of glucose, 10.0 g/L of peptone, and 20.0 g/L of agar were mixed and autoclaved at 115° C. for 20 min), and cultivated at 30° C. in the dark for 10 d.

(2) A special Brey liquid medium for inducing oogonia was prepared as follows: 2.8 g/L of a yeast extract, 2.4 g/L of glucose, 3.2 g/L of a wheat germ powder, and 0.1 mL/L of cottonseed oil were mixed to obtain a first composition. 5.0 g of a cottonseed meal and 100 mL of distilled water were mixed for 1 h at room temperature (25° C.), and then filtered through a cheesecloth to obtain a second composition. 100 mL of the second composition was added to 900 mL of the first composition to obtain the Brey liquid medium.

(3) 100 mL of the Brey medium prepared in the step (2) was dispensed in a 200 mL flask, autoclaved, and cooled. About 0.1 mL of sterile cottonseed oil was added to the flask. A fungal mass from the egg yolk+milk agar medium in the step (1) was inoculated into the flask and cultivated at 30° C. and 150 rpm in the dark on a rotary shaker. The generation of oogonia was observed under a microscope every 3 d continuously for 4 weeks.

### Induced Cultivation of Sporangia and Zoospores:

A spore induction solution was prepared as follows: 66.04 g of (NH₄)₂HPO₄, 68.05 g of KH₂PO₄, and 87.09 g of K₂HPO₄ were added to 500 mL of water to obtain a first solution. 18.38 g of CaCl₂·2H₂O and 25.42 g of MgCl₂ 6H₂O were added to 250 ml of water to obtain a second solution. The first solution and the second solution were autoclaved separately and stored at room temperature until use to obtain a sterilized first solution and a sterilized second solution. 0.5 mL of the sterilized first solution, 0.1 mL of the sterilized second solution, and 1.0 L of sterile distilled water were mixed to obtain the spore induction solution.

Hyphae produced after cultivation in the Brey liquid medium for 3.5 d (from the cultivation in the step (3)) were mixed with 50 mL of the spore induction solution, and cultivated at 30° C. for 6 h to 10 h, during which the development of sporangia and zoospores was observed under a microscope every 30 min.

2. Identification of Lagenidium sp. NL03

### Morphological Identification:

4 mm×4 mm agar pieces were cut off from 2% SDA and observed under a microscope to determine hyphal structures. Morphological characteristics of sporangia and zoospores generated at 30° C. on 2% SDA under induction of the spore induction solution were described. The induced sporangia and zoospores were taken, and 5 μL of 0.02% thimerosal was added to block the movement of zoospores for easy observation.

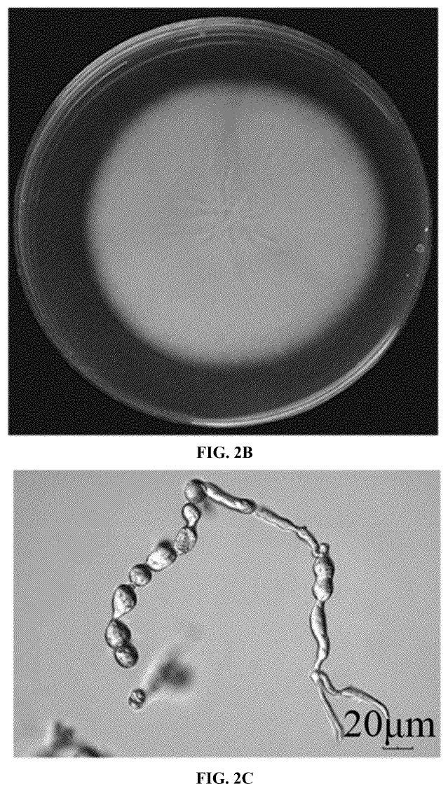

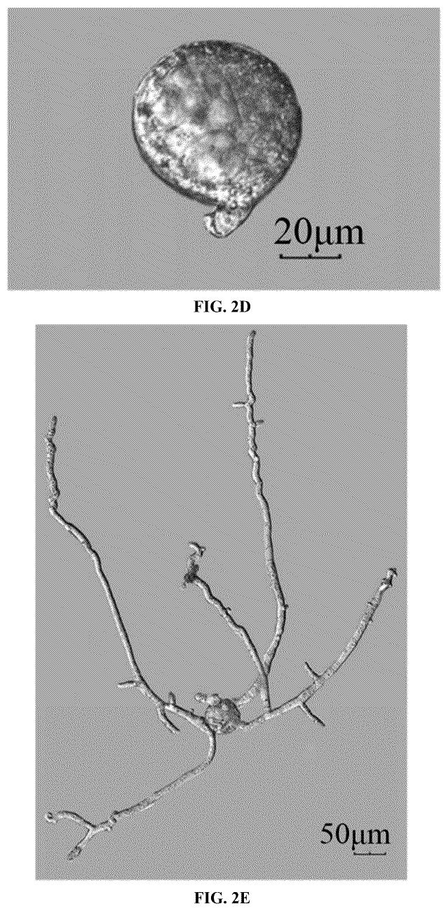

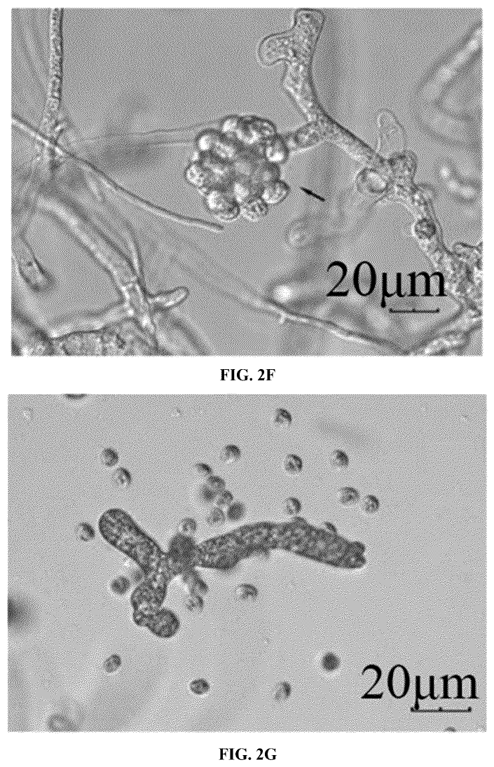

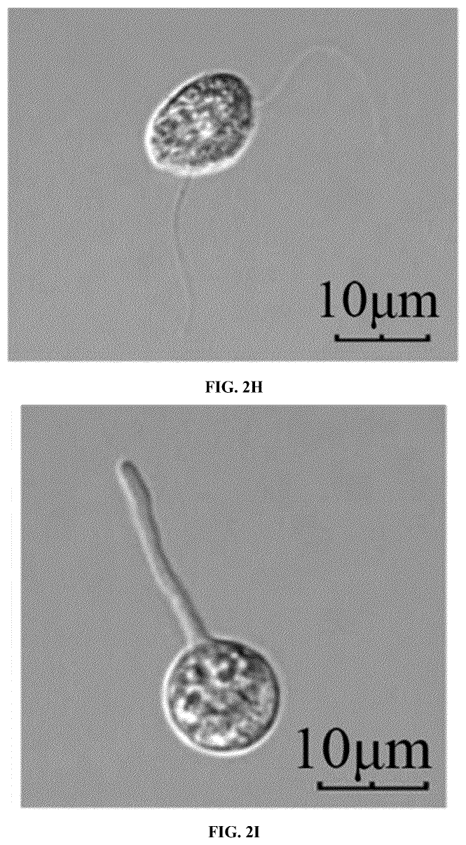

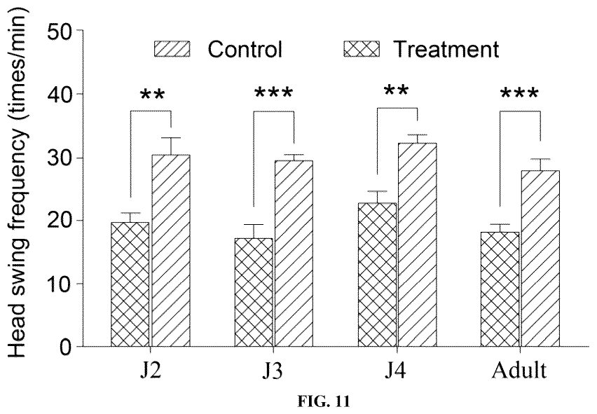

Observation results of the mycelial morphology of Lagenidium sp. NL03 were shown in FIGS. 2A-21. FIG. 2A and FIG. 2B show front and back growth statuses of the strain on 2% SDA, respectively. FIG. 2C shows spherical hyphae in a chain of typical Lagenidium juracyae. FIG. 2D shows broken spherical hyphae. FIG. 2E shows the germination of a spherical hypha. FIG. 2F shows an induced sporangium, where an arrow shows a transparent sporangium wall. FIG. 2G shows zoospores released after sporangia rupture. FIG. 2H shows a zoospore with two flagella under a 100× microscope. FIG. 2I shows the re-germination of a spore to produce a germ tube after flagella of a zoospore fade.

On 2% SDA, Lagenidium sp. NL03 grew into colorless or pale-yellow colonies, there were basically no aerial hyphae, and these colonies were irregularly radial and had undulating wrinkles. Hyphae were septate or non-septate hyphae, and were specialized to produce spherical chain segments. Spores in broken spherical hyphae could continue to germinate to produce new hyphae. The production of sporangia could be induced with the spore induction solution, and after the induction, sporangia with a diameter of 35 μm to 50 μm were produced from the cytoplasm and nucleus through a transparent outlet tube. Each sporangium had 30 to 40 kidney-shaped amphitrichous zoospores of 10 μm, and active zoospores were encapsulated by a transparent sporangium wall. After zoospores were mature, the zoospores broke through the sporangium wall, were released, and then further swam for about 30 min, and were finally encapsulated to produce spherical spores. Germ tubes were gradually germinated out of the spores to produce slender hyphae.

When the generation of oogonia was observed continuously for 4 weeks, it was found that it failed to develop oogonia or other sexual structures in the medium.

### Sequence Alignment and Phylogenetic Analysis:

The strain was inoculated in a 2% SDA liquid medium and cultivated for 72 h at 30° C. on a rotary shaker at 150 rpm. After the cultivation was completed, mycelia were separated through filtration, transferred to a mortar, and ground in the presence of liquid nitrogen.

DNA of the strain was extracted with the MiniBEST Universal Genomic DNA Extraction Kit Ver.5.0 of Takara. Primers for PCR were as follows:

ITS:
(SEQ ID NO: 1)
ITS1 = 5′-TCCGTAGGTGAACCTGCGG-3′
and
(SEQ ID NO: 2)
ITS4 = 5′-TCCTCCGCTTATTGATATGC-3′.
COXII:
(SEQ ID NO: 3)
COX2F = 5′-GGCAAATGGGTTTTCAAGATCC-3′
and
(SEQ ID NO: 4)
COX2R = 5′-CCATGATTAATACCACAAATTTCACTAC-3′.
HSP90:
(SEQ ID NO: 5)
HSP90.LagF = 5′-CAACCTBGGHACSATYGCCAAG-3′
and
(SEQ ID NO: 6)
HSP90.LagR = 5′-ACRAAMGACARGTAYTCVGGCA-3′.
β-TUBB:
(SEQ ID NO: 7)
TUB.LagF = 5′-GGTGGTGGTACCGGTTC-3′
and
(SEQ ID NO: 8)
TUB.LagR = 5′-GACACACGCTTGAACATC-3′.

Thermal cycling parameters for PCR were as follows: denaturation at 95° C. for 10 min, subsequent cycling for 1 min, annealing at 60° C. for 1 min, and extension at 72° C. for 2 min, with 40 repeated cycles; and final extension at 72° C. for 2 min, and incubating for 7 min. A product was sent to Hangzhou Youkang Biotechnology Co., Ltd. for sequencing.

Base sequences of the ITS, COXII, HSP90, and β-TUBB obtained were as follows:

ITS:
(SEQ ID NO: 9)
CATGTGAACCGTTCTACTTGTTTTGTGCGCGACTGCGAAAGCAGAAGTGC
TGAACGAAGGTTAATGTTGGCGCAAGCTAGCATTGGCTGATGAACTTTTA
AACCCCTTTATACCTAATTACTGATAAATACTCTAGGAACGAAAGTTCTT
AGTTTTAATTAGACAACAACTTTCAGCAGTGGATGTCTAGGCTCGCACAT
CGATGAAGAACGCTGCGAACTGCGATACGTAATGCGAATTGCAGAATTCA
GTGAGTCATCGAAATTTTGAACGCATATTGCACTTCCGGGTTATGCCTGG
GAGTATGCCTGTATCAGTGTCCGTAAAACAAACTTGCCTTTCTTTTTCTG
TGTAGTCAGGAATGGAAATGTGCAGAATGTGAAGTGTCTCGAATTACTAT
TATTAATAGCGATTTGAGTCCTTTTAAATCGACACTATCTCTCTATACTG
TTGCTGTGATTGCTTTGCATGAACGCGGTGATCTGCTGATCGTTTGCGCA
TGTTGGCGACTTCGGTAAGAACATATAGGGGAAACCTCAATTTGCGGTAT
GTTGTGGCTTCGGCTCGACAATCTTGCTTATTGTGTGTGGACTCTGTTTT
TATTCGAGGTGTACTAGCGTGTGTGGGCTTGAACGGTGGTTTTGCTGTTT
ATTAGTGTATTGTGACCTGTGACGCATGATCTCTTCGGAGTTTGTGTTCT
AGAGAAATAGGCCAGTTGGGAAACCAATACTTTGACTT.
COXII:
(SEQ ID NO: 10)
ATCATGATTTAATATTTTTTTTAATAATAGTAACTGTTTTTGTTTGTTGG
ATGTTATTTAGAGTTATCACTCTTTTTGATGAAAAAAAAAATCCAATTTC
AGCTACATTTGTACACGGTGCTACTATTGAAATTATTTGGACTACTATTC
CTGCTTTAATTTTATTAACTGTAGCTGTTCCATCTTTTGCTTTATTATAT
TCAATGGATGAAGTTATTGATCCAGTTATTACTTTAAAAGTTATAGGTAG
TCAATGGTATTGGACATATGAATATTCTGATAATTTAGAATATGCAGATG
AACCTTTAATTTTTGATAGCTATATGATACAAGAAGAAGACTTAGAAATA
GGTCAATTAAGATTATTAGAAGTAGATAATCGTATTGTTGTTCCAACTAA
TACACATATTAGAGTATTAATTACAGCATCAGATGTTTTACATTCATGGG
CTGTACCTTCATTAGGTGTTAAATTAGATGCTTGTCCAGGTCGTTTAAAT
CAAACTTCAATGTTTAT;
HSP90:
(SEQ ID NO: 11)
CGGCGCTGACATCAGCATGATCGGTCAGTTCGGTGTGGGTTTCTACTCGG
CTTACCTCGTTGCCGACAAGGTCGTCGTGCACTCGAAGCACAACGATGAC
GAGCAGTACGTCTGGGAGTCGGCTGCCGGTGGCTCGTTCACCGTCACGGC
GGACCACTCGGAACCAATTGCCCGCGGCACCAAGATTGTGCTCAAGCTGA
AGGAGGACATGCTTGAGTACTTGGAGGAGCGCAAGCTCAAGGACCTCGTC
AAGAAGCACTCCGAGTTCATTGGCTTCCCAATCAAGCTCTACGTCGAGAA
GACGGAAGAGAAGGAAGTCACCGATGACGAAGAAGATGAGGACGAGAAGG
AAGGCGAAGATGATAAGCCAACCGTCGAGGAAGTCACCGAGGAGGAGGGT
GACAAGAAGAAGAAGACCAAGAAGATCAAGGAGGTCACCCACGACTGGAA
CCACTTGAACAGCCAGAAGCCAATCTGGATGCGCAAGCCAGAGGATGTCA
CCCACGAGGAGTACGCCTCGTTCTACAAGTCGTTGACCAATGACTGGGAG
GAGCACTTGTCGGTGAAGCACTTCTCGGTGGAAGGCCAGCTCGAATTCAA
GGCCTGCTTGTTCACGCCAAAGCGCGCGCCATTCGACATGTTCGAGGGCG
GTGCCAAGAAGAAGGCCAACAACATCAAGCTCTACGTGCGTCG;
and
β-TUBB:
(SEQ ID NO: 12)
GGCAGCTACCACGGTGACTCCGACTTGCAGTTGGAGCGCATCAACGTGTA
CTACAACGAAGCCACCGGTGGTCGCTATGTGCCACGCGCCATTCTGATGG
ATTTGGAGCCAGGTACCATGGACTCTGTCCGTGCCGGCCCGTACGGCCAG
CTCTTCCGTCCAGACAACTTCGTGTTCGGTCAGACTGGTGCCGGTAACAA
CTGGGCCAAGGGTCACTACACTGAAGGTGCTGAATTGATCGATTCGGTCC
TGGACGTGGTCCGCAAGGAAGCCGAAAGCTGCGATTGCCTGCAAGGTTTC
CAGATCACCCACTCGCTTGGTGGTGGTACCGGTTCGGGTATGGGCACCCT
TCTCATCTCGAAGATTCGTGAAGAGTACCCAGATCGTATCATGTGCACGT
ACTCGGTCTGCCCGTCGCCAAAGGTGTCGGATACCGTCGTTGAGCCATAC
AACGCCACACTCTCGGTGCATCAGCTTGTCGAAAACGCCGATGAAGTCAT
GTGCTTGGACAACGAAGCCCTGTACGATATCTGCTTCCGTACGCTCAAGC
TCACGACCCCAACCTACGGTGACTTGAACCACTTGGTGTGCGCTGCCATG
TCTGGTATCACCACGTGCTTGCGTTTCCCTGGTCAGCTCAACTCCGACTT
GCGCAAGCTTGCCGTGAACTTGATTCCGTTCCCACGTCTCCACTTCTTCA
TGATTGGTTTCGCCCCATTGACCTCGCGCGGCTCCCAGCAGTACCGTGCG
TTGACGGTCCCAGAATTGACCCAGCAGCAGTTCGATGCTAAGAACATGAT
GTGCGCTGCCGATCCTCGTCACGGTCGTTACCTGACGGCTGCATGCATGT
TCCGTGGTCGCATGAGCACCAAGG.

The phylogenetic and molecular evolution analysis was conducted for the strain with MEGA7 (http://www.megasoftware.net). The ITS and COXII sequences of Lagenidium sp. NL03 investigated were aligned with the corresponding sequences of other Lagenidium, Myzocytiopsis, Pythium, Paralagenidium, and Globisporangium registered by the National Center for Biotechnology Information (NCBI). Achlyaambisexualis and Saprolegnia ferax were selected as outgroups to construct phylogenetic trees by a neighbor-joining (NJ) method. The phylogenetic trees each were tested 1,000 times with Bootstrap. MTLA-06Paralagenidium_karlingi was selected as an outgroup, a command module was added at an end of each dataset (the command module for constructing a maximum parsimony (MP) evolutionary tree based on PAUP: BEGIN PAUP; outgroup1MTLA_06_Paralagenidium_karlingi; Set criterion=parsimony; Bootstrap nreps=1000keepall; contree; describetree 1/plot=both brlens=yes; savetrees from=1to=1000; END), and the MP method was adopted to conduct multi-gene coalescent tree inference for ITS, COXII, HSP90, and Tubulin sequences of Lagenidium sp. NL03 based on PAUP4.

TABLE 1
Strains selected for multi-gene coalescent tree
inference and NCBI registration numbers thereof

| Taxonomic name | Strain ID | ITS | COXII | HSP90 | β-TUBB |
|---|---|---|---|---|---|
| Lagenidium humanum | ATCC 76726 | JX970867 | AF290310 | JX999094 | JX999125 |
| Lagenidium sp. | MTLA-01 | JF919611 | JX970853 | JX999097 | JX999127 |
| Lagenidium giganteum | MTLA-10 | KJ506125 | KJ506083 | KJ506097 | KJ506111 |
| Lagenidium giganteum | MTLA-12 | KJ506129 | KJ506084 | KJ506098 | KJ506112 |
| Lagenidium giganteum | MTLA-14 | KJ506127 | KJ506085 | KJ506099 | KJ506113 |
| Lagenidium giganteum | MTLA-15 | KJ506126 | KJ506087 | KJ506103 | KJ506116 |
| Lagenidium giganteum | MTLA-16 | KJ506130 | KJ506086 | KJ506102 | KJ506115 |
| Lagenidium giganteum | MTLA-17 | KJ506131 | KJ506088 | KJ506101 | KJ506117 |
| Lagenidium giganteum | MTLA-18 | KJ506128 | KJ506089 | KJ506100 | KJ506114 |
| Paralagenidium karlingii | MTLA-06 | JX970885 | JX970841 | JX999112 | JX999143 |
| Lagenidium ajelloi | MTLA-20 | KJ506135 | KJ506094 | KJ506109 | KJ506124 |
| Lagenidium ajelloi | MTLA-21 | KJ506137 | KJ506095 | KJ506108 | KJ506122 |
| Lagenidium ajelloi | MTLA-22 | KJ506138 | KJ506096 | KJ506107 | KJ506123 |
| Lagenidium albertoi | MTLA-13 | JX970870 | JX970838 | JX999115 | JX999142 |
| Lagenidium vilelae | MTLA-24 | KJ506133 | KJ506091 | KJ506105 | KJ506119 |
| Lagenidium vilelae | Tiss25 | KJ506132 | KJ506090 | KJ506104 | KJ506118 |
| Lagenidium sp. | CO1-MO133 | JX970876 | JX970860 | JX999109 | JX999135 |
| Lagenidium sp. | CO3-BS40 | JX970882 | JX970866 | JX999106 | JX999141 |
| Lagenidium sp. | COO-AP | JX970879 | JX970861 | JX999105 | JX999136 |
| Lagenidium sp. | C99-MC | JX970875 | JX970859 | JX999104 | JX999134 |
| Lagenidium sp. | C99-AW | JX970874 | JX970858 | JX999103 | JX999133 |
| Lagenidium sp. | C99-SM | JX970873 | JX970857 | JX999102 | JX999132 |
| Lagenidium sp. | C99-PC | JX970872 | JX970856 | JX999101 | JX999131 |
| Lagenidium sp. | CO2-SW136 | JX970878 | JX970863 | JX999111 | JX999138 |
| Lagenidium sp. | CO3-PP08 | JX970880 | JX970865 | JX999108 | JX999140 |

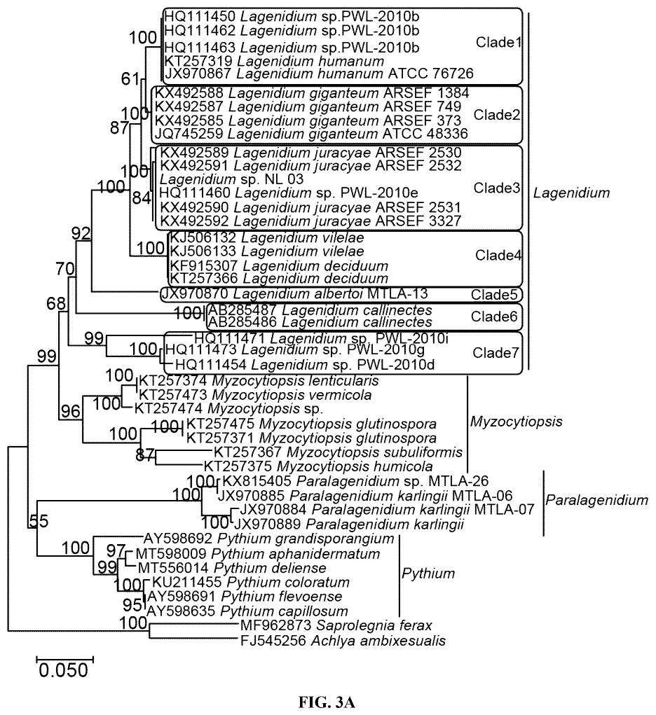

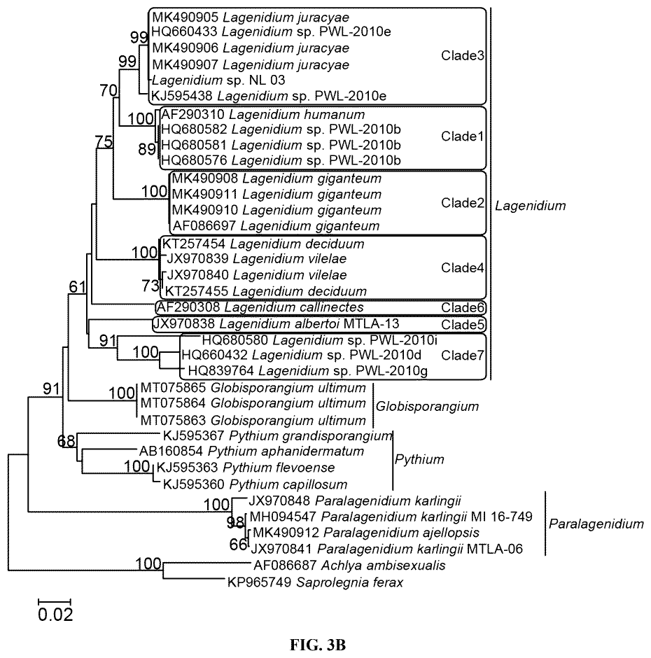

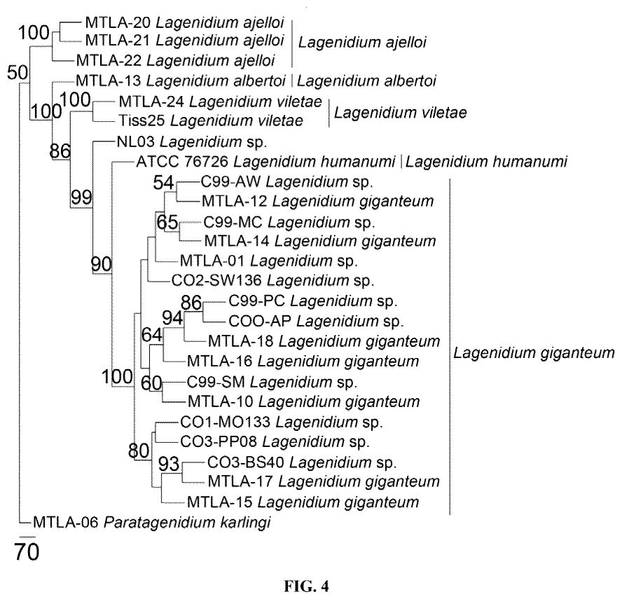

The phylogenetic trees constructed according to the ITS and COXII sequences of the strain were shown in FIGS. 3A-3B. FIG. 3A shows a phylogenetic tree constructed according to the ITS sequence of Lagenidium sp. NL03 and MEGA7 by the NJ method, and FIG. 3B shows a phylogenetic tree constructed according to the COXII sequence of Lagenidium sp. NL03 and MEGA7 by the NJ method. A result of the multi-gene coalescent tree inference for ITS, COXII, HSP90, and β-TUBB sequences of the strain was shown in FIG. 4. According to the results, with Saprolegniaferax and Achlya ambixesualis as outgroups, ITS was aligned with the sequences of Lagenidium, Myzocytiopsis, Paralagenidium, and Pythium, COXII was aligned with the sequences of Lagenidium, Globisporangium, Pythium, and Paralagenidium, and a phylogenetic tree was calculated and constructed with MEGA7 by the NJ method. The new sequences obtained in the present application were marked in bold. A node support rate ≥50% was represented by a number, and a branch length corresponded to a corrected distance. The tree construction results showed that the test strain Lagenidium sp. NL03 had the most close genetic relationship to Lagenidium juracyae in terms of ITS and COXII sequences.

The multi-gene coalescent tree inference was conducted for ITS and three exons (COXII, HSP90, and β-TUBB) of the strain and other Lagenidium by the MP method based on PAUP4. It was found that the strain belonged to the genus Lagenidium. Because Lagenidium juracyae with the most close genetic relationship to the strain lacked sequencing results of corresponding genes, Lagenidium sp. NL03 became a separate branch in the multi-gene coalescent tree inference and was closely related to Lagenidium humanum evolutionarily.

The strain isolated and identified in this example was Lagenidium juracyae with a strain number of NL03, which was deposited in the China General Microbiological Culture Collection Center (CGMCC) at NO. 1 West Beichen Road, Chaoyang District, Beijing, China on May 8, 2023, with an accession number CGMCC NO. 40587.

### Example 3 Antagonism and Nematode Attraction Experiment for Lagenidium sp. NL03

B. cinerea is an “edible fungus” to cultivate B. xylophilus. Therefore, in this experiment, it was investigated whether there was an antagonism relationship between Lagenidium sp. NL03 and B. cinerea and whether Lagenidium sp. NL03 and B. cinerea each had an attraction or rejection response to B. xylophilus.

In this experiment, the following four treatments were set: First to third treatment groups: a B. cinerea fungal mass, a Lagenidium sp. NL03 fungal mass, and an agar piece of 1 cm were inoculated to a center of a water agar medium, respectively. A fourth treatment group: A 1 cm Lagenidium sp. NL03 fungal mass and a B. cinerea fungal mass were symmetrically inoculated to two ends of a water agar medium, where a distance between the two fungal masses was 5 cm and each fungal mass was 1 cm away from an edge of a Petri dish.

For each of the above four treatment groups, three replicates were set, and the cultivation was conducted at 25° C. in the dark for 24 h, which was convenient for hyphae to release exudative substances. Then, 10 μL of a B. xylophilus suspension (including about 150 nematodes) was added to a position 2 cm away from a fungal mass on a plate in the first to third treatment groups, and 10 μL of a nematode suspension was added to a midmost position between the two fungal masses in the fourth treatment group. Each sealed plate was incubated at 25° C. for 24 h in the dark, and then a fungal mass was gently picked with a teasing needle and observed under a stereo-microscope to count a number of nematodes swimming to the fungal mass.

Like the fourth treatment group, the two fungal masses were inoculated on 2% SDA without nematodes and co-cultivated at 25° C. in the dark for 7 d, and it was observed whether there was an antagonistic effect between the two strains.

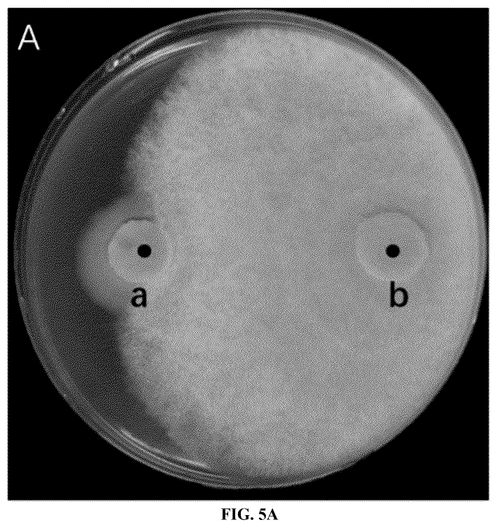

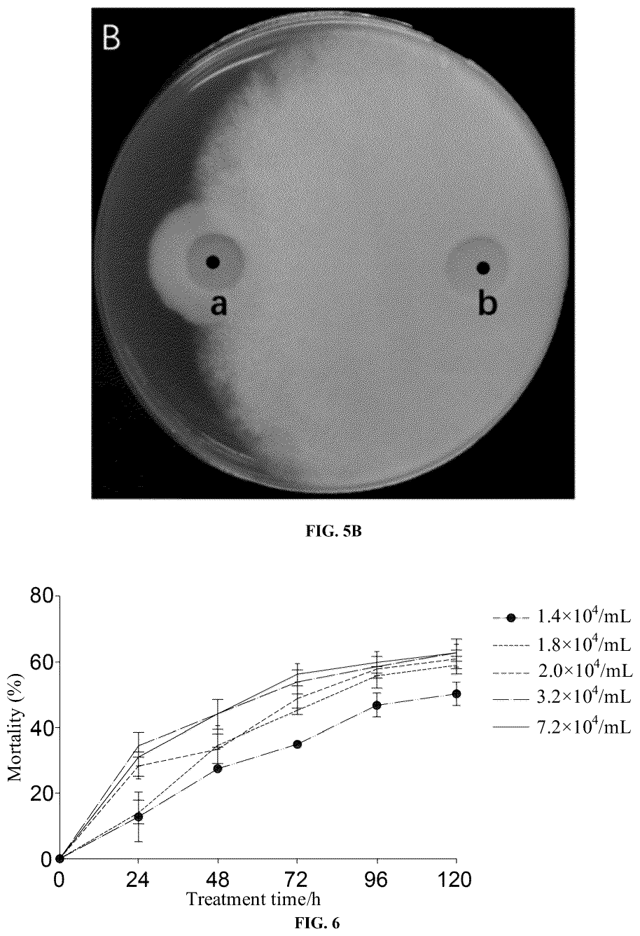

The antagonism results between Lagenidium sp. NL03 and B. cinerea were shown in FIGS. 5A-5B. In FIGS. 5A-5B, A shows a front side of the medium, B shows a back side of the medium, a indicates the Lagenidium sp. NL03 fungal mass, and b indicates the B. cinerea fungal mass. The results showed that the two strains both grew obviously after being cultivated on 2% SDA for 7 d, but a growth rate of B. cinerea was significantly higher than a growth rate of Lagenidium sp. NL03, and hyphae of B. cinerea covered hyphae of Lagenidium sp. NL03, indicating that there was no antagonistic effect between the two strains.

Attraction results of Lagenidium sp. NL03 and B. cinerea for nematodes within 24 h were shown in Table 2:

TABLE 2
Numbers of nematodes attracted by Lagenidium
sp. NL03 and B. cinerea within 24 h

| | | | B. cinerea/ | Lagenidium sp. | |
|---|---|---|---|---|---|
| | | Lagenidium | Lagenidium sp. | NL03/ | Agar |
| | B. cinerea | sp. NL03 | NL03 | B. cinerea | piece |
| | B. cinerea | Lagenidium | B. cinerea/ | Lagenidium sp. | Agar |
| | | sp. NL03 | Lagenidium sp. | NL03/ | piece |
| | | | NL03 | B. cinerea | |
| B. xylophilus | 51 ± 12a | 2 ± 0.5c | 42 ± 6a | 2 ± 0.5c | 27 ± 11b |

Notes:
The data in the table are expressed as mean ± standard deviation. Different letters for data of a same row indicate that there is a significant difference at the level of P < 0.05 according to the Tukey test. The party in bold in the table is a counted party in the fourth treatment group.

It can be seen from the comparison of significant differences in the table that there are significant differences among the groups. Compared with the agar piece in the control group, B. cinerea has a significant attraction to B. xylophilus, while Lagenidium sp. NL03 exhibits not only no attraction, but also repulsion to B. xylophilus. It is speculated that Lagenidium sp. NL03 emits a specified substance to affect the tropism of B. xylophilus.

A plurality of examples were provided below for nematocidal tests of Lagenidium sp. NL03:

After nematodes are completely infected by Lagenidium sp. NL03, contents of nematodes will be re-used as nutrients for the growth of hyphae or the re-generation of zoosporangia to release spores to infect other nematodes. As a result, a completely-infected nematode will be cannibalized to leave merely a transparent body wall layer. The transparent body wall may be mistaken as a mycelium or ignored during observation and statistics, which affects the calculation of a mortality of nematodes. Therefore, in this experiment, each number of surviving nematodes is counted to calculate a corrected mortality (%)=[(x−y)/x]×100, where x represents a survival rate in a control group and y represents a survival rate in a treatment group.

### Example 4 Infection of Spore Suspensions of Lagenidium sp. NL03 at Different Concentrations for B. xylophilus

Spore suspensions produced after cultivation for 6 h to 10 h (preferably 8 h) in the induced sporangium and zoospore cultivation step in Example 2 were taken. Because there were different numbers of zoospores induced in different flasks, a plurality of flasks were taken to calculate spore concentrations in these flasks during this experiment. Nematodes on each medium were isolated by a Baermann funnel. In a 96-well plate, about 100 B. xylophilus nematodes were mixed with 200 μL of a spore solution in each well. In a control group, a spore induction solution without spores was added. Three replicates were set for each bottle. The 96-well plate was incubated at 25° C. The continuous observation was conducted for 5 d, and the death of B. xylophilus was recorded.

The infection results of spore suspensions of Lagenidium sp. NL03 at different concentrations for B. xylophilus were shown in FIG. 6. Different numbers of spores were released by the spore induction solution in different flasks, and a spore suspension with a high concentration could not be obtained by low-speed centrifugation-based enrichment. Thus, in this experiment, spore solutions of 5 concentrations were adopted to infect B. xylophilus, so as to evaluate a nematocidal ability of Lagenidium sp. NL03.

According to the 5 d continuous observation, a mortality of B. xylophilus increased rapidly at 24 h to 72 h, but increased slowly at 72 h to 120 h. It could be reasonably speculated that the mortality of nematodes increased slowly because flagella of some zoospores faded and spores losing a swimming ability settled at a bottom and thus were in relatively reduced contact with nematodes. When a concentration of a spore suspension increased from 1.4×10⁴/mL to 3.2×10⁴/mL, a nematocidal ability of Lagenidium sp. NL03 increased with the increase of the concentration. When the concentration of the spore suspension increased from 3.2×10⁴/mL to 7.2×10⁴/mL, the nematocidal ability of Lagenidium sp. NL03 did not change significantly. According to experiments, under the existing spore collection conditions, when a spore concentration of Lagenidium sp. NL03 was 3.2×10⁴/mL or more, the nematocidal ability of Lagenidium sp. NL03 was stabilized at about 60%.

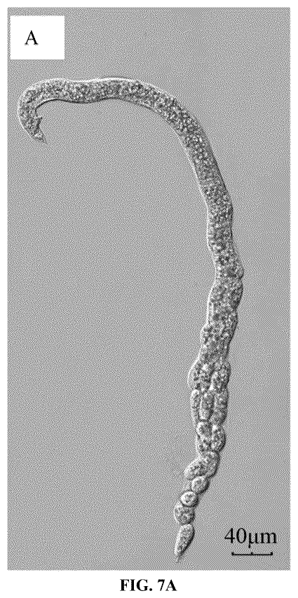

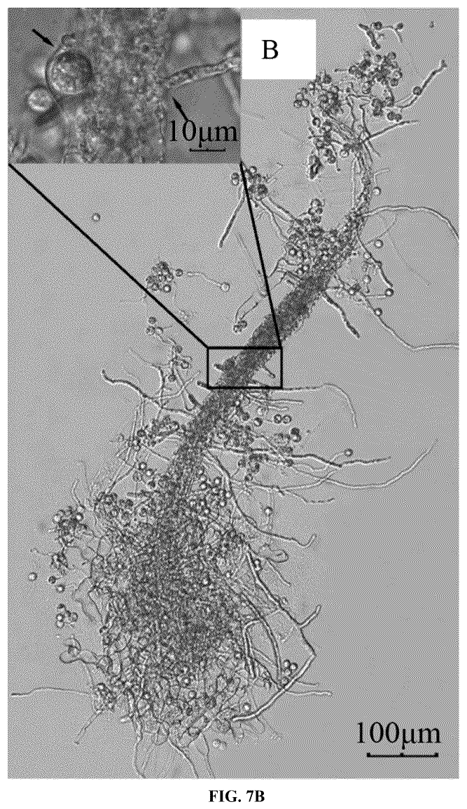

Images illustrating the infection of Lagenidium sp. NL03 into B. xylophilus were shown in FIGS. 7A-7B. In FIGS. 7A-7B, A shows a morphology of B. xylophilus at an initial stage of infection, B shows a morphology B. xylophilus at a final stage of infection, and the arrow in B refers to a germ tube germinated after Lagenidium sp. NL03 is infected into B. xylophilus. After B. xylophilus was infected by Lagenidium sp. NL03, it was observed that spores of Lagenidium sp. NL03 were re-generated in the nematode and extended outward through a body wall of the nematode to produce a new discharge tube. During the experiment, it was observed that infecting hyphae in a host were irregular septate hyphae, and after the hyphae were mature, a transparent outlet tube was re-formed and new zoosporangia were produced in vitro to release spores. When B. xylophilus was completely infected by Lagenidium sp. NL03, contents of B. xylophilus would be re-used as nutrients for the growth of hyphae or the re-generation of zoosporangia to release spores to infect other nematodes. A completely-infected nematode would be cannibalized to leave merely a transparent body wall layer.

In the experiment, it was observed that zoospores did not infect all B. xylophilus nematodes, and the selection of zoospores for B. xylophilus tended to be random. Many spores may adhere to a B. xylophilus nematode, while no spore may adhere to the adjacent nematode. When spores had adhered to a body surface of a nematode, the nematode may also twist and rub to make the spores separated from the body surface of the nematode, resulting in the failed infection into the nematode.

### Example 5 Infection of Lagenidium sp. NL03 for Other Nematodes

Aphelenchoides besseyi (A. besseyi), Caenorhabditis elegans (C. elegans), Bursaphelenchus mucronatus (B. mucronatus), and B. xylophilus each were isolated from a medium by the same method. In a 24-well plate, about 100 nematodes were mixed with 200 μL of a spore solution (including about 8×10³ spores) in each well. Three replicates were set for each treatment. The 24-well plate was incubated at 25° C. The continuous observation was conducted for 3 d, and the death of nematodes was recorded.

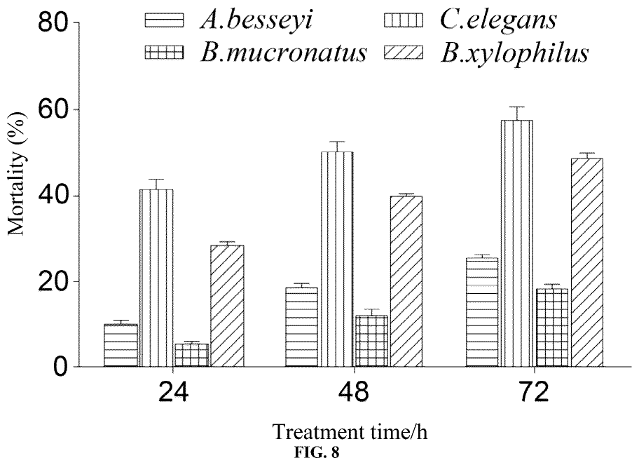

The infection results of Lagenidium sp. NL03 for other nematodes were shown in FIG. 8. A. besseyi, C. elegans, B. mucronatus, and B. xylophilus each were infected with a spore suspension of Lagenidium sp. NL03. It could be known that the spores of Lagenidium sp. NL03 exhibited an ability to infect all of the four nematodes, and exhibited the strongest ability to infect the soil nematode C. elegans, which was also consistent with the isolation of Lagenidium sp. NL03 from the soil nematode. However, Lagenidium sp. NL03 exhibited a very weak ability to infect B. mucronatus, an allied species of B. xylophilus. It was speculated that substances secreted by B. mucronatus and B. xylophilus affected the adhesion and infection of spores of Lagenidium sp. NL03 or Lagenidium sp. NL03 exhibited a specific recognition ability for nematodes to be infected.

### Example 6 Investigation of Impacts of a Spore Suspension and a Cytoplasmic Extract on the Hatching of B. xylophilus Eggs

Hyphae growing on a 2% SDA plate after 7 d of cultivation were collected with tweezers, transferred to a mortar, and ground in the presence of liquid nitrogen, sterile water was added, and centrifugation was conducted to obtain a supernatant as a cytoplasmic extract. B. xylophilus produced after 3 d of cultivation was isolated from a plate, added to a glass dish, and allowed to lay eggs for 1 h, and a resulting supernatant was then poured off to obtain synchronized eggs left at a bottom of the glass dish. The eggs at the bottom were gently collected with a silicone knife. About 1,000 eggs were taken and mixed with each of 1 mL of a spore solution (including 8×10³ spores) and 1 mL of the cytoplasmic extract. Three replicates were set for each treatment. The cultivation was conducted at 25° C. for 24 h, and then a number of unhatched eggs was recorded to calculate a hatching rate.

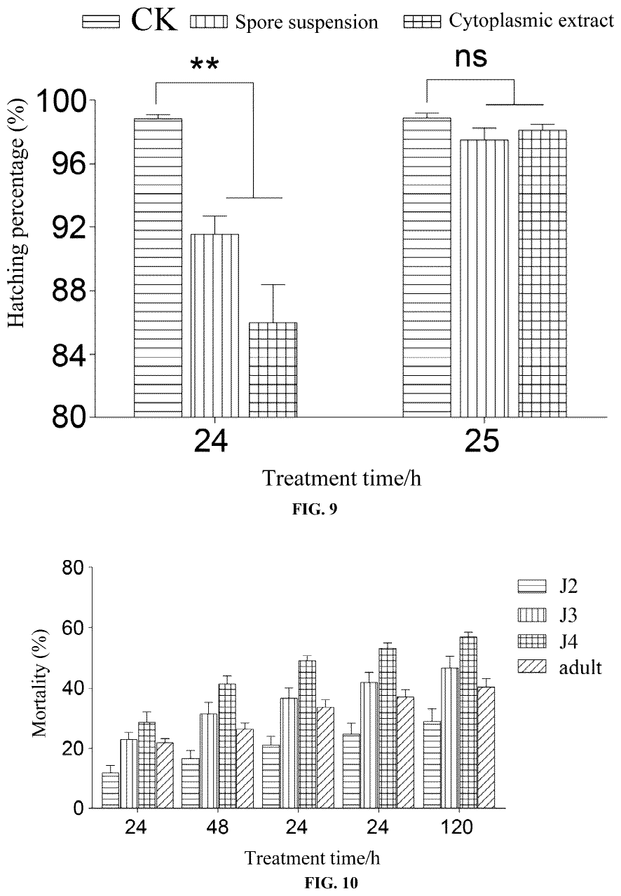

The impacts of the spore suspension (a spore suspension produced after cultivation for 6 h to 10 h (preferably 8 h) in the induced sporangium and zoospore cultivation step in Example 2) and the cytoplasmic extract on the hatching of B. xylophilus eggs were shown in FIG. 9. The eggs of B. xylophilus usually hatched after being placed in clear water at 25° C. for 24 h. Times required for the eggs to hatch after being mixed with the spore suspension and the cytoplasmic extract of Lagenidium sp. NL03 were observed and compared to determine the impacts of the spore suspension and the cytoplasmic extract on the B. xylophilus eggs. According to experiments, at 24 h, hatching rates of eggs treated with the spore suspension and the cytoplasmic extract were significantly different from a hatching rate of eggs in a control group, indicating that there were specified substances in the spore suspension and cytoplasmic extract treatments to inhibit the hatching of eggs. At 25 h, there was no difference in hatching rates between the control group and the two treatment groups, indicating that an inhibitory effect was weakened and eggs could hatch normally. In the subsequent experiments, it was tried to determine whether the cytoplasmic extract of Lagenidium sp. NL03 could kill B. xylophilus, but the cytoplasmic extract basically did not have an ability to kill nematodes, which was not described in detail.

### Example 7 Investigation of Infection of a Spore Suspension for B. xylophilus at Different Instars

Synchronized eggs were acquired by the same method as in Example 6. The eggs were incubated for 24 h to hatch into 2nd instar larvae, and then inoculated on a PDA plate covered with B. cinerea. Nematodes were collected at 24 h, 48 h, and 72 h to obtain 3rd instar larvae, 4th instar larvae, and adult nematodes, respectively. In a 24-well plate, about 100 synchronized instar nematodes were mixed with 200 μL of a spore solution (including about 3.6×10³ spores) in each well. Six replicates were set for each instar. The 24-well plate was incubated at 25° C. The continuous observation was conducted for 5 d, and the death of B. xylophilus was recorded.

The infection of the spore suspension for B. xylophilus at different instars was shown in FIG. 10. When B. xylophilus was infected with Lagenidium sp. NL03, it was observed that there were a large number of zoospores near some nematodes, especially nematodes at large instars, while there were a small number of zoospores near some nematodes. Therefore, it was speculated that, before infection, zoospores of Lagenidium sp. NL03 would select nematodes, especially nematodes at different instars. Therefore, this idea was verified by treating B. xylophilus at different instars. The results showed that Lagenidium sp. NL03 indeed exhibited selectivity for nematodes during infection. 4th instar larvae that were almost mature were infected the most, while small 2nd instar larvae and adult nematodes that were fully developed were infected less.

### Example 8 Investigation of Impacts of a Spore Suspension on the Motility of B. xylophilus at Different Instars

After synchronized nematodes were infected for 24 h, 1 μL of a resulting nematode suspension was collected, placed on a glass slide, diluted with clear water, and observed under a microscope to determine the motility of nematodes. A head swing frequency of B. xylophilus within 1 min was counted. The above process was repeated 3 times, where 1 μL was collected each time, and head swing frequencies of 3 to 5 nematodes in a droplet were counted each time.

The impacts of the spore suspension on the motility of B. xylophilus at different instars were shown in FIG. 11. After B. xylophilus was treated with the spore suspension of Lagenidium sp. NL03, B. xylophilus at different instars exhibited significant differences from a control group in terms of the motility, and had greatly-reduced head swing frequencies, indicating that the infection of Lagenidium sp. NL03 reduced a viability of B. xylophilus, or Lagenidium sp. NL03 produced a substance for inhibiting a viability of B. xylophilus to make B. xylophilus inert, which was conducive for the infection of spores.

The above examples are merely some implementations of the present application, and the description thereof is specific and detailed, but should not be construed as limiting the patent scope of the present application. It should be noted that those of ordinary skill in the art can further make several variations and improvements without departing from the concept of the present application, and these variations and improvements all fall within the protection scope of the present application. Therefore, the protection scope of the present application should be subject to the protection scope defined by the claims.

What is claimed is:

1. Lagenidium juracyae with a Bursaphelenchus xylophilus (B. xylophilus)-killing activity, wherein the Lagenidium juracyae has a strain number of NL03 and an accession number of CGMCC NO. 40587.

2. A biocontrol agent for killing B. xylophilus, comprising a spore suspension of the Lagenidium juracyae according to claim 1 as an active ingredient.

3. A preparation method of the biocontrol agent according to claim 2, comprising:
activating the Lagenidium juracyae to obtain activated Lagenidium juracyae, transferring the activated Lagenidium juracyae to an egg yolk+milk agar medium, and cultivating to produce mycelia; transferring a part of the mycelia to a Brey liquid medium for inducing oogonia, and further cultivating for 2.5 d to 4 d to obtain a hypha solution; and mixing the hypha solution with a spore induction solution, and cultivating at 28° C. to 30° C. for 6 h to 10 h.

4. A control method for B. xylophilus, comprising: impregnating the B. xylophilus with a spore suspension of the Lagenidium juracyae according to claim 1 or applying the spore suspension of the Lagenidium juracyae according to claim 1 to a forest tree.

5. The control method according to claim 4, wherein the spore suspension is a spore suspension when zoospores are released and swim.

6. The control method according to claim 4, wherein the B. xylophilus is a 3rd to 4th instar larva.

7. The control method according to claim 4, wherein the B. xylophilus is a 4th instar larva.

8. The control method according to claim 4, wherein a concentration of the spore suspension is 1×10⁴/mL to 10×10⁴/mL; and the impregnating is conducted for 24 h to 120 h.


SEQUENCE LISTING (12 sequences)

SEQ ID NO: 1 - DNA, 19 bp, synthetic construct
  misc_feature 1..19: note = primer ITS1
  source 1..19: mol_type = other DNA
tccgtaggtgaacctgcgg

SEQ ID NO: 2 - DNA, 20 bp, synthetic construct
  misc_feature 1..20: note = sequence of primer ITS4
  source 1..20: mol_type = other DNA
tcctccgcttattgatatgc

SEQ ID NO: 3 - DNA, 22 bp, synthetic construct
  misc_feature 1..22: note = sequence of primer COX2F
  source 1..22: mol_type = other DNA
ggcaaatgggttttcaagatcc

SEQ ID NO: 4 - DNA, 28 bp, synthetic construct
  misc_feature 1..28: note = sequence of primer COX2R
  source 1..28: mol_type = other DNA
ccatgattaataccacaaatttcactac

SEQ ID NO: 5 - DNA, 22 bp, synthetic construct
  misc_feature 1..22: note = sequence of primer HSP90.LagF
  source 1..22: mol_type = other DNA
caacctbgghacsatygccaag

SEQ ID NO: 6 - DNA, 22 bp, synthetic construct
  misc_feature 1..22: note = sequence of primer HSP90.LagR
  source 1..22: mol_type = other DNA
acraamgacargtaytcvggca

SEQ ID NO: 7 - DNA, 17 bp, synthetic construct
  misc_feature 1..17: note = sequence of primer TUB.LagF
  source 1..17: mol_type = other DNA
ggtggtggtaccggttc

SEQ ID NO: 8 - DNA, 18 bp, synthetic construct
  misc_feature 1..18: note = sequence of primer TUB.LagR
  source 1..18: mol_type = other DNA
gacacacgcttgaacatc

SEQ ID NO: 9 - DNA, 738 bp, Lagenidium juracyae
  misc_feature 1..738: note = sequence of ITS of Lagenidium juracyae
  source 1..738: mol_type = other DNA
catgtgaaccgttctacttgttttgtgcgcgactgcgaaagcagaagtgctgaacgaaggttaatgttggcgcaagctagcattggctgatgaacttttaaacccctttatacctaattactgataaatactctaggaacgaaagttcttagttttaattagacaacaactttcagcagtggatgtctaggctcgcacatcgatgaagaacgctgcgaactgcgatacgtaatgcgaattgcagaattcagtgagtcatcgaaattttgaacgcatattgcacttccgggttatgcctgggagtatgcctgtatcagtgtccgtaaaacaaacttgcctttctttttctgtgtagtcaggaatggaaatgtgcagaatgtgaagtgtctcgaattactattattaatagcgatttgagtccttttaaatcgacactatctctctatactgttgctgtgattgctttgcatgaacgcggtgatctgctgatcgtttgcgcatgttggcgacttcggtaagaacatataggggaaacctcaatttgcggtatgttgtggcttcggctcgacaatcttgcttattgtgtgtggactctgtttttattcgaggtgtactagcgtgtgtgggcttgaacggtggttttgctgtttattagtgtattgtgacctgtgacgcatgatctcttcggagtttgtgttctagagaaataggccagttgggaaaccaatactttgactt

SEQ ID NO: 10 - DNA, 517 bp, Lagenidium juracyae
  misc_feature 1..517: note = exon COXII
  source 1..517: mol_type = other DNA
atcatgatttaatattttttttaataatagtaactgtttttgtttgttggatgttatttagagttatcactctttttgatgaaaaaaaaaatccaatttcagctacatttgtacacggtgctactattgaaattatttggactactattcctgctttaattttattaactgtagctgttccatcttttgctttattatattcaatggatgaagttattgatccagttattactttaaaagttataggtagtcaatggtattggacatatgaatattctgataatttagaatatgcagatgaacctttaatttttgatagctatatgatacaagaagaagacttagaaataggtcaattaagattattagaagtagataatcgtattgttgttccaactaatacacatattagagtattaattacagcatcagatgttttacattcatgggctgtaccttcattaggtgttaaattagatgcttgtccaggtcgtttaaatcaaacttcaatgtttat

SEQ ID NO: 11 - DNA, 693 bp, Lagenidium juracyae
  misc_feature 1..693: note = exon HSP90
  source 1..693: mol_type = other DNA
cggcgctgacatcagcatgatcggtcagttcggtgtgggtttctactcggcttacctcgttgccgacaaggtcgtcgtgcactcgaagcacaacgatgacgagcagtacgtctgggagtcggctgccggtggctcgttcaccgtcacggcggaccactcggaaccaattgcccgcggcaccaagattgtgctcaagctgaaggaggacatgcttgagtacttggaggagcgcaagctcaaggacctcgtcaagaagcactccgagttcattggcttcccaatcaagctctacgtcgagaagacggaagagaaggaagtcaccgatgacgaagaagatgaggacgagaaggaaggcgaagatgataagccaaccgtcgaggaagtcaccgaggaggagggtgacaagaagaagaagaccaagaagatcaaggaggtcacccacgactggaaccacttgaacagccagaagccaatctggatgcgcaagccagaggatgtcacccacgaggagtacgcctcgttctacaagtcgttgaccaatgactgggaggagcacttgtcggtgaagcacttctcggtggaaggccagctcgaattcaaggcctgcttgttcacgccaaagcgcgcgccattcgacatgttcgagggcggtgccaagaagaaggccaacaacatcaagctctacgtgcgtcg

SEQ ID NO: 12 - DNA, 874 bp, Lagenidium juracyae
  misc_feature 1..874: note = exon -TUBB
  source 1..874: mol_type = other DNA
ggcagctaccacggtgactccgacttgcagttggagcgcatcaacgtgtactacaacgaagccaccggtggtcgctatgtgccacgcgccattctgatggatttggagccaggtaccatggactctgtccgtgccggcccgtacggccagctcttccgtccagacaacttcgtgttcggtcagactggtgccggtaacaactgggccaagggtcactacactgaaggtgctgaattgatcgattcggtcctggacgtggtccgcaaggaagccgaaagctgcgattgcctgcaaggtttccagatcacccactcgcttggtggtggtaccggttcgggtatgggcacccttctcatctcgaagattcgtgaagagtacccagatcgtatcatgtgcacgtactcggtctgcccgtcgccaaaggtgtcggataccgtcgttgagccatacaacgccacactctcggtgcatcagcttgtcgaaaacgccgatgaagtcatgtgcttggacaacgaagccctgtacgatatctgcttccgtacgctcaagctcacgaccccaacctacggtgacttgaaccacttggtgtgcgctgccatgtctggtatcaccacgtgcttgcgtttccctggtcagctcaactccgacttgcgcaagcttgccgtgaacttgattccgttcccacgtctccacttcttcatgattggtttcgccccattgacctcgcgcggctcccagcagtaccgtgcgttgacggtcccagaattgacccagcagcagttcgatgctaagaacatgatgtgcgctgccgatcctcgtcacggtcgttacctgacggctgcatgcatgttccgtggtcgcatgagcaccaagg